(19) United States
(12) Patent Application Publication
(10) Pub. No.: US 2005/0226816 A1
(43) Pub. Date: Oct. 13, 2005

(54) Detection of transmembrane potentials using asymmetric thiobarbituric acid-derived polymethine oxonols

(75) Inventors: Zhenjun Diwu, Sunnyvale, CA (US); Yong Yao, N Potomac, MD (US); Jianjun He, Sunnyvale, CA (US); Guobing Xiang, Santa Clara, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: AnaSpec, Inc., San Jose, CA (US)

(21) Appl. No.: 10/971,311

(22) Filed: Oct. 21, 2004

Related U.S. Application Data
(60) Provisional application No. 60/514,347, filed on Oct. 24, 2003.

(51) Int. Cl. A61B 5/00; A61B 8/00; A61B 10/00; A61B 5/05

(52) U.S. Cl. 424/9.6

(57) ABSTRACT

The present invention relates generally to the detection and measurement of transmembrane potentials using an asymmetric thiobarbituric acid-derived polymethine oxonol (shown below). In particular, the present invention is directed to compositions and optical methods for determining transmembrane potentials across the plasma membrane of biological cells using a moderately hydrophobic asymmetric thiobarbituric acid-derived polymethine oxonols. The method comprises a moderately hydrophobic asymmetric thiobarbituric acid-derived polymethine oxonol anion capable of redistributing from a first face of the membrane to a second face of the membrane in response to changes in the potential of the membrane. In one aspect the method is used to identify compounds which modulate membrane potentials in biological membranes. 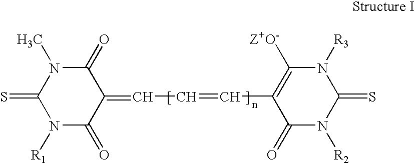 wherein R1, R2, and R3 are (a) independently selected from the group consisting of hydrogen, alkyl, haloalkyl and heteroalkyl, and (b) R1, R2 and R3 are not simultaneously methyl; n is an integer from 1 to 3; Z is Na, K, ammonium or other biologically acceptable salt.

This application claims priority under 35 U.S.C. § 119 to U.S. Provisional Application No. 60/514,347 entitled Detection of Transmembrane Potentials using Asymmetric Thiobarbituric Acid Derived Polymethine Oxonols and filed on Oct. 24, 2003.

### BACKGROUND OF THE INVENTION

1. Field of the Invention

The present invention relates generally to the detection and measurement of transmembrane potentials using an asymmetric thiobarbituric acid-derived polymethine oxonol. In particular, the present invention is directed to compositions and optical methods for determining transmembrane potentials across the plasma membrane of biological cells using a moderately hydrophobic asymmetric thiobarbituric acid-derived polymethine oxonols. The method comprises a moderately hydrophobic asymmetric thiobarbituric acid-derived polymethine oxonol anion capable of redistributing from a first face of the membrane to a second face of the membrane in response to changes in the potential of the membrane. In one aspect the method is used to identify compounds which modulate membrane potentials in biological membranes.

2. Background of the Art

The plasma membrane of a cell typically has a transmembrane potential of approximately −70 mV (negative inside) as a consequence of K⁺, Na⁺ and Cl⁻ concentration gradients that are maintained by active transport processes. Increases and decreases in membrane potential (referred to as membrane hyperpolarization and depolarization, respectively) play a central role in many physiological processes, including nerve-impulse propagation, muscle contraction, cell signaling and ion-channel gating [Shapiro HM. “Cell membrane potential analysis.” Methods Cell Biol 41, 121-133 (1994); Baxter D F, Kirk M, Garcia A F, Raimondi A, Holmqvist M H, Flint K K, Bojanic D, Distefano P S, Curtis R, Xie Y. “A novel membrane potential-sensitive fluorescent dye improves cell-based assays for ion channels.” J Biomol Screen 7, 79 (2002); Falconer M, Smith F, Surah-Narwal S, Congrave G, Liu Z, Hayater P, Ciaramella G, Keighley W, Haddock P, Waldron G, Sewing A. “High-throughput screening for ion channel modulators” J Biomol Screen 7, 460 (2002)]. In general, there are two distinct methods to measure cell membranes, (a) direct electrical measurement of cell membrane potentials, e.g, the so-called ‘Patch Clamping’ technique, and (b) indirect optical sensing of membrane potentials using a membrane potential-sensitive dye as an indicator. Fluorescence detection and imaging of cellular electrical activity is a technique of great importance [Grinvald, A., Frostig, R. D., Lieke, E., and Hildesheim, R. “Optical imaging of neuronal activity.” Physiol. Rev. 68, 1285-1366 (1988); Salzberg, B. M. “Optical recording of electrical activity in neurons using molecular probes.” In Current Methods in Cellular Neurobiology. J. L. Barker (editor) Wiley, New York. 1983, pp 139-187; Cohen, L. B. and S. Lesher. “Optical monitoring of membrane potential: methods of multisite optical measurement.” In Optical Methods in Cell Physiology. P. de Weer and B. M. Salzberg (editors), 1985, Wiley, New York. pp 71-99]. The optical method that uses a fluorescent indicator has steadily gained popularity in recent years due to its convenience, high throughput and improved sensitivity. Potentiometric probes are a critical factor in the optical measurement of membrane potentials. The existing potentiometric probes include the cationic or zwitterionic styryl dyes, the cationic carbocyanines and rhodamines, the anionic oxonols and hybrid oxonols and merocyanine 540. The class of dyes determines factors such as accumulation in cells, response mechanism and toxicity. The fluorescent indicators used in the optical measurement of membrane potential have been traditionally divided into two classes:
(1) Fast-response dyes: These dyes are usually cell-impermeable and have fast response to changes in membrane potentials because they need little or no translocation. [Loew, L. M., “How to choose a potentiometric membrane probe”, In Spectroscopic Membrane Probes. CRC Press, Boca Raton L., 1988, pp 139-151; Loew, L. M., “Potentiometric membrane dyes”, In Fluorescent and Luminescent Probes for Biological Activity. W. T. Mason (editor), Academic Press, San Diego, 1993, pp 150-160]. However, they are insensitive because they sense the electric field with only a part of a unit charge moving less than the length of the molecule, which in turn is only a small fraction of the distance across the membrane. Furthermore, a significant fraction of the total dye signal comes from molecules that sit on irrelevant membranes or cells and that dilute the signal from the few correctly placed molecules.

(2) Slow-response dyes: In contrast to the above-mentioned ‘fast-response’ dyes, these dyes are usually hydrophobic and cell-permeable. They are quite sensitive although they have a slow redistribution of permeant ionized dyes from the extracellular medium into the cell. The ratio of their concentrations between the inside and outside of the cell can change by up to the Nernstian limit of 10 fold for a 60 mV change in transmembrane potential. However, for the permeable ions to establish new equilibria, the dye ions must diffuse through unstirred layers in each aqueous phase and the low-dielectric-constant interior of the plasma membrane. These processes result in their slow responses to changes in membrane potentials. Moreover, such dyes distribute into all available hydrophobic binding sites indiscriminately. Therefore, selectivity between cell types is difficult. Additionally, any additions of hydrophobic proteins or reagents to the external solution, or changes in exposure to hydrophobic surfaces, are prone to cause artifacts.

In view of the above drawbacks of existing fluorescent dyes used in optical measurement of membrane potentials, improved methods and compositions are needed to detect small variations in transmembrane potentials with a rapid response and strong fluorescence signal, preferably on a millisecond to second timescale. Also needed are methods and compositions less susceptible to the effects of changes in external solution composition. The critical factors to develop such membrane potential detection technologies are the effective design and synthesis and testing/screening of membrane potential-sensitive fluorescent dyes. This invention fulfils this and related needs.

The thiobarbituric acid-based oxonols, often referred to as “DiSBAC” dyes (in the case of symmetric thiobarbituric acid-derived polymethine oxonols) form a family of spectrally distinct potentiometric probes with excitation maxima covering most range of visible wavelengths. DiSBAC₂(3) has been the most popular oxonol dye for membrane potential measurement [Plasek J, Sigler K. “Slow fluorescent indicators of membrane potential: a survey of different approaches to probe response analysis.” J Photochem Photobiol B 33, 101-124 (1996); Loew L M. “Characterization of Potentiometric Membrane Dyes.” Adv Chem Ser 235, 151 (1994); Loew, L. M., “How to choose a potentiometric membrane probe”, In Spectroscopic Membrane Probes. CRC Press, Boca Raton L., 1988, pp 139-151; Loew, L. M., “Potentiometric membrane dyes”, In Fluorescent and Luminescent Probes for Biological Activity. W. T. Mason (editor), Academic Press, San Diego, 1993, pp 150-160]. These dyes enter depolarized cells where they bind to intracellular proteins or membranes and exhibit enhanced fluorescence. Increased depolarization results in more influx of the anionic dye and thus an increase in fluorescence.

In general, DiSBAC dyes bearing longer alkyl chains had been proposed to have better properties for measuring membrane potentials [Loew L M., “Potentiometric Membrane Dyes”. In Fluorescent and Luminescent Probes for Biological Activity, Mason W T, 2.sup.nd Ed. 1999, pp 210-221; Gonzalez J E, Tsien R Y. “Improved indicators of cell membrane potential that use fluorescence resonance energy transfer.” Chem Biol 4, 269-277 (1997)]. Recently this hypothesis has been disputed by the fact that DiSBAC₁(3) possess better properties for optical measurement of membrane potentials than DiSBAC₂(3) (U.S. Patent Application 20030087332). In this invention, DiSBAC₆(3) and DiSBAC₀(3) are prepared to confirm the existing theories of designing effective fluorescent indicators for measuring membrane potentials. Neither of the compounds prove to be better fluorescent indicators for measuring membrane potentials than DiSBAC₁(3) although DiSBAC₀(3) or DiSBAC₆(3) would have been a better fluorescent membrane potential indicator [than DiSBAC₁(3)] according to U.S. Patent Application 20030087332 or according to the generally accepted hypothesis that more hydrophobic oxonols tend to be better fluorescent membrane potential indicators.

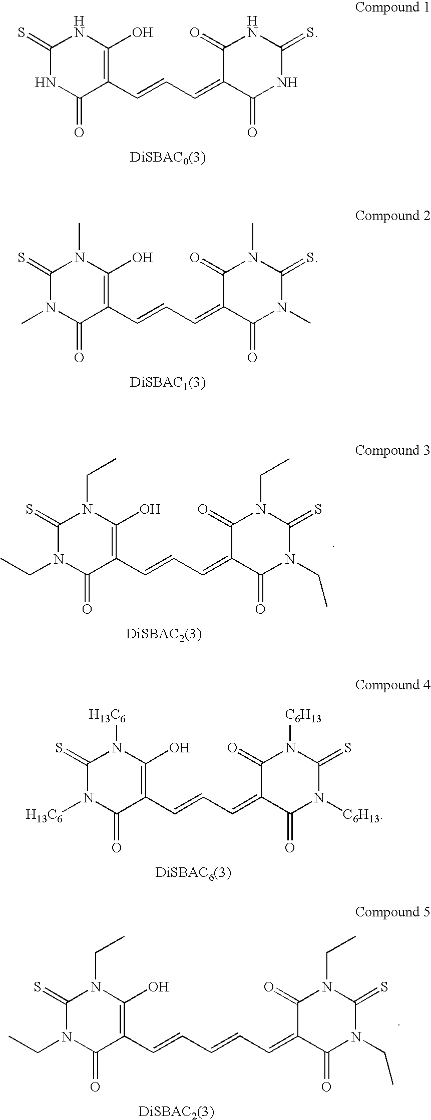

In this invention, we have discovered that the thiobarbituric acid-derived polymethine oxonols with moderate hydrophobicity tend to be sensitive fluorescent indicators for optical measurement of membrane potentials, and to be less prone to effects of extracellular environmental changes, e.g. culture medium and temperature etc. The substitutes on the nitrogen atoms of two thiobarbituric acid moieties need be critically fine-tuned. The existing thiobarbituric acid-derived polymethine oxonols used in optical measurement of membrane potentials are symmetric oxonols that are referred as ‘DiSBAC’ and have the four same alkyl groups on the two thiobarbituric acid moieties. The symmetric oxonols are generally prepared as shown in FIG. 2. This synthetic approach is ill-adapted to prepare asymmetric thiobarbituric acid-derived polymethine oxonols for screening, optimization and selection of good fluorescent thiobarbituric acid-derived polymethine oxonols for optical measurement of membrane potentials. This invention provides an improved method to prepare asymmetric thiobarbituric acid-derived polymethine oxonols for optical measurement of membrane potentials as shown in FIG. 3. By this improved synthetic method, the substituents of thiobarbituric acid-derived polymethine oxonols can be readily fine-tuned to give the best optimized properties for optical measurement of membrane potentials.

### SUMMARY OF THE INVENTION

Conventional electrophysiological techniques (the so called ‘Patch Clamping’ recording) use an electrode to measure membrane potentials. The electrical method is not only invasive, but also limited to measurement of membrane potentials in a single cell. By contrast, the optical indicators described herein are particularly advantageous for simultaneously monitoring the membrane potential of a population of cells, e.g., many neurons or muscle cells. Optical indicators, unlike conventional microelectrodes, do not require physical puncture of the membrane. In many cells or organelles, such puncture is highly injurious or mechanically difficult to accomplish although some automated ‘Patch Clamping’ technologies have been developed in recent years. The optical indicators are still most suitable for cells too small or fragile to be impaled by electrodes.

This invention provides improved optical methods and compositions for determining transmembrane electrical potential (membrane potential), particularly across the outermost (plasma) membrane of living cells. In one aspect, the method comprises: (a) contacting a fluorescent thiobarbituric acid-derived polymethine oxonol with cells. The fluorescent indicator is capable of redistributing from a first face of the membrane to a second face of the membrane in response to changes in the potential of the membrane (as determined by the Nernst equation); (b) exposing the membrane-containing structure to excitation light of an appropriate wavelength, typically in the ultraviolet or visible region; and (c) Relating the fluorescence intensity to the membrane potential.

In another aspect of the invention, the voltage sensing methods allow one to detect the effect of test samples, such as potential therapeutic drug molecules, on the activation/deactivation of ion transporters (channels, pumps, or exchangers) embedded in the membrane.

In another aspect of the invention, an improved method is developed to prepare asymmetric thiobarbituric acid-derived polymethine oxonols for optical measurement of membrane potentials. This new synthetic approach provides an improved method to readily fine-tune substituents of thiobarbituric acid-derived polymethine oxonols that give the best optimized properties for optical measurement of membrane potentials.

### BRIEF DESCRIPTION OF THE DRAWINGS

The invention may be better understood with reference to the accompanying drawings.

FIG. 1. The chemical structures of DiSBAC₀(3), DiSBAC₁(3), DiSBAC₂(3), DiSBAC₆(3) and DiSBAC₂(5).

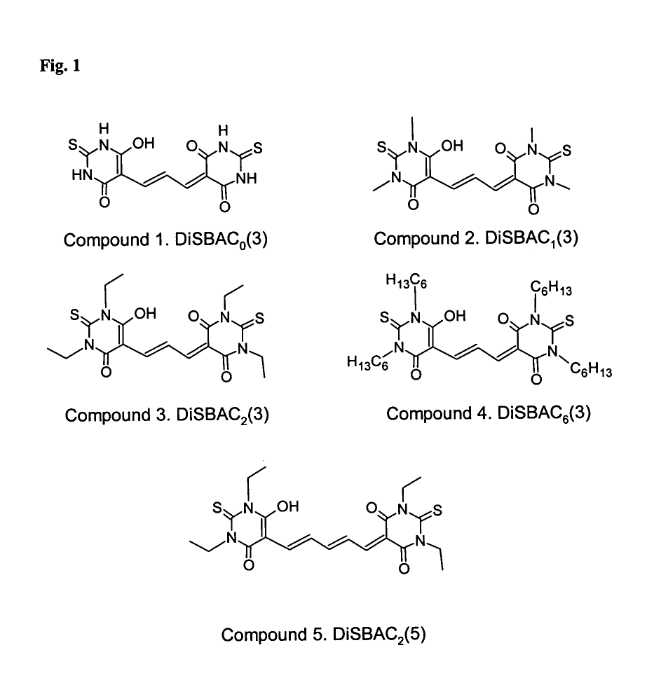

FIG. 2. The typical synthesis of symmetric thiobarturic acid-derived polymethine oxonols.

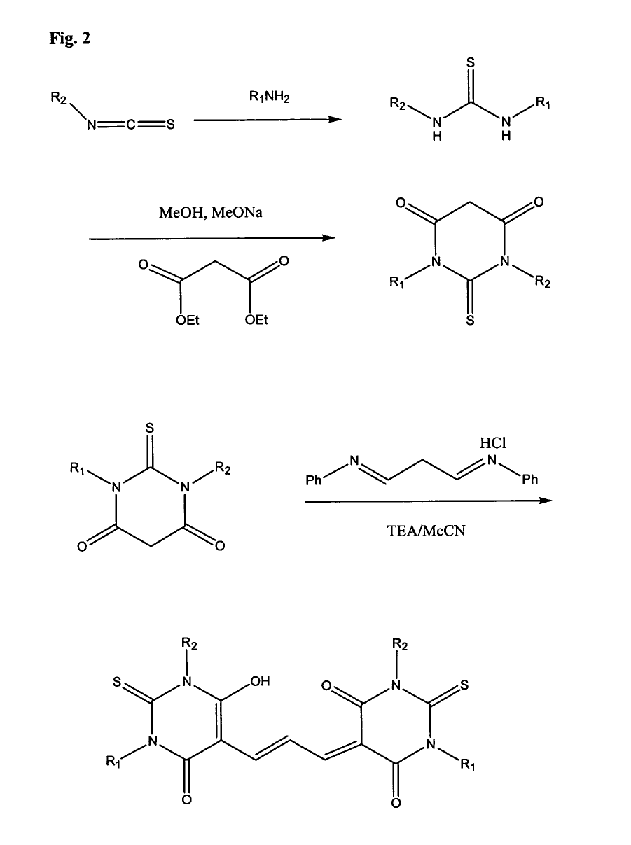

FIG. 3. The improved synthesis of asymmetric thiobarbituric acid-derived polymethine oxonols that have different substitutents on the nitrogen atoms.

FIG. 4. The absorption and fluorescence spectra of Compound 18, a representative asymmetric thiobarbituric acid-derived polymethine oxonol.

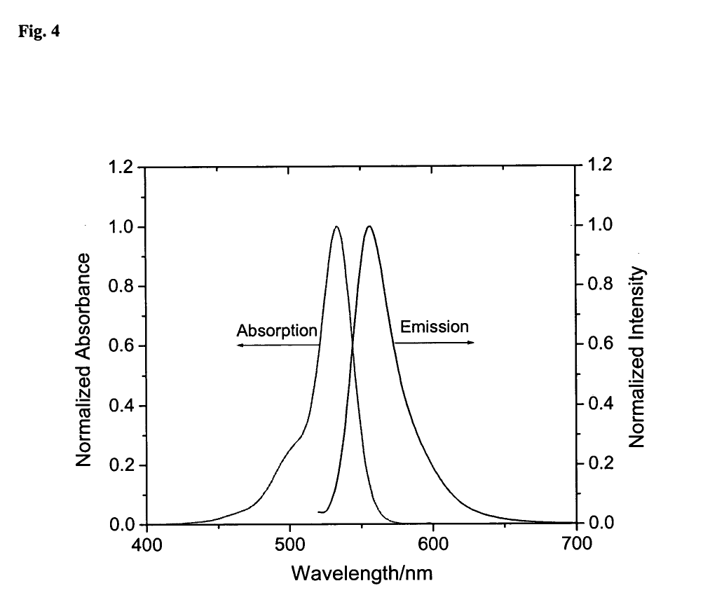

FIG. 5. The fluorescence comparison of Compound 18 (a representative asymmetric thiobarbituric acid-derived polymethine oxonol) in HBSS buffer and octanol;

FIG. 6. The HEK293 cell line that stably incorporates a CNG channel mutant gene is prepared, and then loaded with Compound 18 (a representative asymmetric thiobarbituric acid-derived polymethine oxonol) by incubating the cells for 2 hours at room temprature with the dye at 5 μM, in combination with a membrane-impermeable non-fluorescent dye to quench extracellular fluorescence. The CNG channels are activated by cyclic nucleotides (cGMP and cAMP) whereby conducting cation currents are carried by mixed ions Na⁺, K⁺ and Ca²⁺, and the changes in intracellular cyclic nucleotide concentration are coupled to membrane potential changes. The kinetic responses are recorded on FLIPR (Molecular Devices Corp., Sunnyvale, Calif.). Activation of one endogenous G protein-coupled adenosine A2B receptor is detected by measuring membrane depolarization mediated through activation of CNG channels (see FIG. 6). Addition of NECA agonist at 3 μM (marked with an arrow in this figure) affords the increase in fluorescence intensity of Compound 18 by about 4 fold.

FIG. 7. A stable HEK293 cell line expressing one CNG channel mutant is used in the assays. CNG channel is activated by NECA in a dose-dependent manner. Endpoint assays are performed in 384-well plates. Cells are loaded with different voltage-sensitive dyes for about 2 hours at room temperature. Fluorescence change expressed in signal over the background (F/Fb) is measured 30 minutes after addition of NECA at various concentrations. Dose-responses of NECA are plotted as shown in this figure. The moderately hydrophobic asymmetric thiobarbituric acid-derived polymethine oxonols of this invention, e.g., Compounds 18 and 20, demonstrate significant improvement in fluorescence signal strength and in quality of assay data over the existing symmetric thiobarbituric acid-derived polymethine oxonols.

### DESCRIPTION OF THE PREFERRED EMBODIMENT

The following definitions are set forth to illustrate and define the meaning and scope of the various terms used to describe the invention herein.

The term “hydrocarbyl” shall refer to an organic radical comprised of carbon chains to which hydrogen and other elements are attached. The term includes alkyl, alkenyl, alkynyl and aryl groups, groups which have a mixture of saturated and unsaturated bonds, carbocyclic rings and includes combinations of such groups. It may refer to straight chain, branched-chain, cyclic structures or combinations thereof.

The term “alkyl” refers to a branched or straight chain acyclic, monovalent saturated hydrocarbon radical of one to twenty carbon atoms. This term is further exemplified by radicals such as methyl, ethyl, n-propyl, i-propyl, n-butyl, i-butyl, sec-butyl, t-butyl, n-pentyl, 2-methyl butyl, 3-methyl butyl, and 2-ethyl propyl.

The term “alkenyl” refers to an unsaturated hydrocarbon radical which contains at least one carbon-carbon double bond and includes straight chain, branched chain and cyclic radicals. This term is further exemplified by radicals such as ethenyl, propenyl, 1-butenyl, 3-methyl-1-butenyl, cyclopentenyl and cyclohexenyl.

The term “alkynyl” refers to an unsaturated hydrocarbon radical which contains at least one carbon-carbon triple bond and includes straight chain, branched chain and cyclic radicals. This term is further exemplified by radicals such as ethynyl, propynyl, 1-butynyl, 2-butynyl, 3-methyl-1-butynyl, and 3-methyl-1-pentynyl.

The term “heteroalkyl” refers to a branched or straight chain acyclic, monovalent saturated radical of two to forty atoms in the chain in which at least one of the atoms in the chain is a heteroatom, such as, for example, oxygen, nitrogen or sulfur. This term is further exemplified by radicals such as OCH₃, NHCH₃, N(CH₃)₂, SCH₃, CH₂OH, CH₂NH₂, CH₂SH, CH₂OCH₃, CH₂NHCH₃, CH₂N(CH₃)₂, CH₂SCH₃, CH₂CH₂OH, CH₂CH₂NH₂, CH₂CH₂NH(CH₃), CH₂CH₂N(CH₃)₂, and CH₂CH₂SH.

The term “cycloalkyl” refers to a monovalent saturated carbocyclic radical of three to twelve carbon atoms in the carbocycle. This term is further exemplified by radicals such as cyclopropyl, cyclobutyl, cyclopentyl, and cyclohexyl.

The term “heterocycloalkyl” refers to a monovalent saturated cyclic radical of one to twelve atoms in the ring, having at least one heteroatom, such as oxygen or sulfur within the ring. This term is further exemplified by radicals such as epoxidyl, aziridinyl, tetrahydrofuranyl, tetrahydropyrroleyl, tetrahydrothiopheneyl, and morpholinyl.

The term “alkylene” refers to a fully saturated, cyclic or acyclic, divalent, branched or straight chain hydrocarbon radical of one to forty carbon atoms. This term is further exemplified by radicals such as methylene, ethylene, n-propylene, 1-ethylethylene, and n-heptylene.

The term “heteroalkylene” refers to an alkylene radical in which at least one of the atoms in the chain is a heteroatom. This term is further exemplified by radicals such as NHCH₂CHCH₂, OCH₂CHCH₂, SCH₂CHCH₂, CH₂NHCH₂CHCH₂, CH₂OCH₂CHCH₂, CH₂SCH₂CHCH₂, CH₂NHCH₂CHCHCH₃, CH₂OCH₂CHCHCH₃, and CH₂SCH₂CHCHCH₃.

The term “substituted phenyl” refers to a phenyl group which is mono-, di-, tri-, or tetra-substituted, independently, with hydrocarbyl, alkyl, lower-alkyl, cycloalkyl or cycloalkyl-lower alkyl. This term is further exemplified by radicals such as PhCH₃, Ph(CH₃)₂, Ph(CH₃)₃, PhC₂H₅, Ph(CH₃)(C₂H₅), Ph(C₂H₅)₂, and PhC₃H₅.

The term “aryl” refers to an aromatic monovalent carbocyclic radical having a single ring (e.g., phenyl) or multiple condensed rings (e.g., naphthyl, anthracenyl), which can optionally be mono-, di-, or tri-substituted, independently, with hydrocarbyl, alkyl, lower-alkyl, cycloalkyl or cycloalkyl lower alkyl.

The term “halo” refers to fluoro, bromo, chloro and iodo.

The term “polymethine oxonol” refers to molecules comprising two potentially acidic groups linked via a polymethine chain and possessing a single negative charge delocalized between the two acidic groups. The preferred acidic groups are asymmetric thiobarbiturates.

The term “asymmetric thiobarbituric acid-derived oxonols” referes to thiobarbituric acid-containing oxonols in which at least one of the substituents on the nitrogen atoms of thiobarbiturate moieties is different from the three other substituents.

The term “moderately hydrophobic” when used in the context of the hydrophobic ion refers to a species whose partition coefficient between a physiological saline solution (e.g. HBSS) and octanol is between 5 and 5000, preferably at least about 20 and 1000. Methods of determining partition coefficients and adsorption coefficients are known to those of skill in the art.

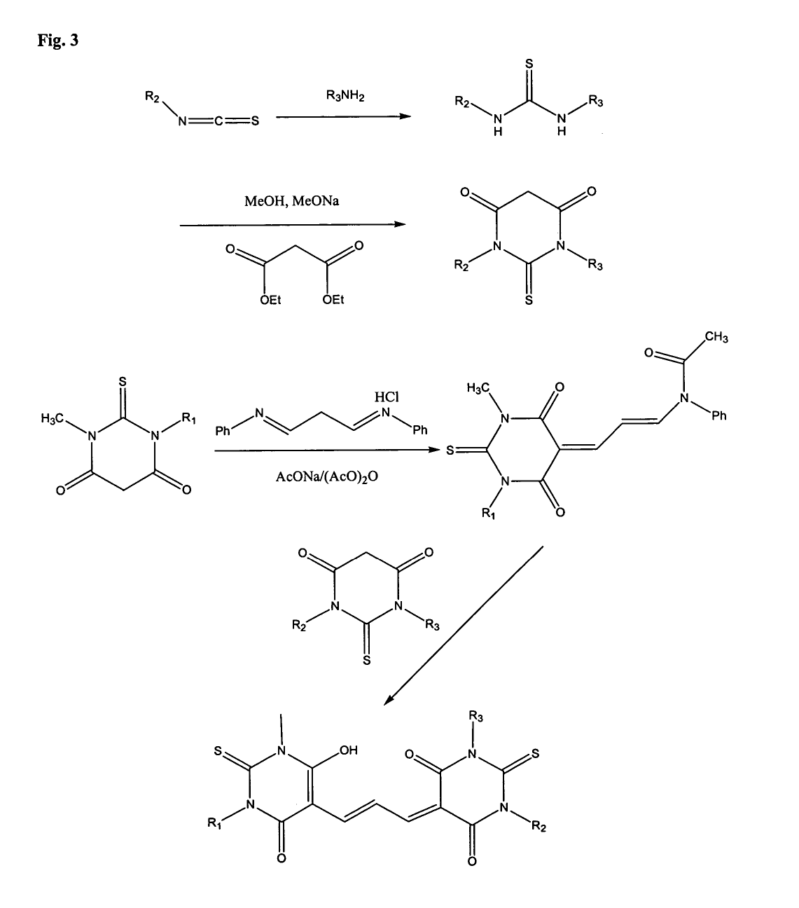

Oxonol compounds used in this invention have a general structure of Formula I. 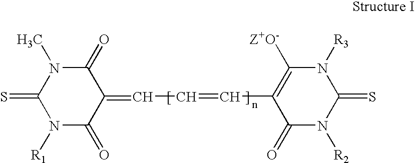
wherein R1, R2, and R3 are (a) independently selected from the group consisting of hydrogen, alkyl, haloalkyl and heteroalkyl, and (b) R1, R2 and R3 are not simultaneously methyl; n is an integer from 1 to 3; Z is Na, K, ammonium or other biologically acceptable salt. Oxonols in which the R groups on a particular thiobarbiturate moiety are different to each other are specifically contemplated by this invention and can be prepared from asymmetrical urea derivatives as described in FIG. 3 via the corresponding intermediates of Structure II. 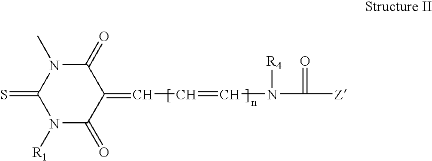
wherein R1 and R4 are independently selected from the group consisting of hydrogen, hydrocarbyl, preferably alkyl, haloalkyl and heteroalkyl; n is an integer from 1 to 3; Z′ is a hydrocarbyl group. Preferably R4 is phenyl, and Z′ is methyl or trifluoromethyl.

Exemplary oxonol compounds of this invention include, without limitation, the following:

Class 1—R1 is hydrogen, R2 is hydrogen, and R3 is hydrogen; R1 is hydrogen, R2 is hydrogen, and R3 is alkyl; R1 is hydrogen, R2 is hydrogen, and R3 is haloalkyl; R1 is hydrogen, R2 is hydrogen, and R3 is heteroalkyl.

Class 2—R1 is hydrogen, R2 is alkyl, and R3 is hydrogen; R1 is hydrogen, R2 is alkyl, and R3 is alkyl; R1 is hydrogen, R2 is alkyl, and R3 is haloalkyl; R1 is hydrogen, R2 is alkyl, and R3 is heteroalkyl.

Class 3—R1 is hydrogen, R2 is haloalkyl, and R3 is hydrogen; R1 is hydrogen, R2 is haloalkyl, and R3 is alkyl; R1 is hydrogen, R2 is haloalkyl, and R3 is haloalkyl; R1 is hydrogen, R2 is haloalkyl, and R3 is heteroalkyl.

Class 4—R1 is hydrogen, R2 is heteroalkyl, and R3 is hydrogen; R1 is hydrogen, R2 is heteroalkyl, and R3 is alkyl; R1 is hydrogen, R2 is heteroalkyl, and R3 is haloalkyl; R1 is hydrogen, R2 is heteroalkyl, and R3 is heteroalkyl.

Class 5—R1 is alkyl, R2 is hydrogen, and R3 is hydrogen; R1 is alkyl, R2 is hydrogen, and R3 is alkyl; R1 is alkyl, R2 is hydrogen, and R3 is haloalkyl; R1 is alkyl, R2 is hydrogen, and R3 is heteroalkyl.

Class 6—R1 is alkyl, R2 is alkyl, and R3 is hydrogen; R1 is alkyl, R2 is alkyl, and R3 is alkyl (excluding methyl); R1 is alkyl, R2 is alkyl, and R3 is haloalkyl; R1 is alkyl, R2 is alkyl, and R3 is heteroalkyl.

Class 7—R1 is alkyl, R2 is haloalkyl, and R3 is hydrogen; R1 is alkyl, R2 is haloalkyl, and R3 is alkyl; R1 is alkyl, R2 is haloalkyl, and R3 is haloalkyl; R1 is alkyl, R2 is haloalkyl, and R3 is heteroalkyl.

Class 8—R1 is alkyl, R2 is heteroalkyl, and R3 is hydrogen; R1 is alkyl, R2 is heteroalkyl, and R3 is alkyl; R1 is alkyl, R2 is heteroalkyl, and R3 is haloalkyl; R1 is alkyl, R2 is heteroalkyl, and R3 is heteroalkyl.

Class 9—R1 is haloalkyl, R2 is hydrogen, and R3 is hydrogen; R1 is haloalkyl, R2 is hydrogen, and R3 is alkyl; R1 is haloalkyl, R2 is hydrogen, and R3 is haloalkyl; R1 is haloalkyl, R2 is hydrogen, and R3 is heteroalkyl.

Class 10—R1 is haloalkyl, R2 is alkyl, and R3 is hydrogen; R1 is haloalkyl, R2 is alkyl, and R3 is alkyl; R1 is haloalkyl, R2 is alkyl, and R3 is haloalkyl; R1 is haloalkyl, R2 is alkyl, and R3 is heteroalkyl.

Class 11—R1 is haloalkyl, R2 is haloalkyl, and R3 is hydrogen; R1 is haloalkyl, R2 is haloalkyl, and R3 is alkyl; R1 is haloalkyl, R2 is haloalkyl, and R3 is haloalkyl; R1 is haloalkyl, R2 is haloalkyl, and R3 is heteroalkyl.

Class 12—R1 is haloalkyl, R2 is heteroalkyl, and R3 is hydrogen; R1 is haloalkyl, R2 is heteroalkyl, and R3 is alkyl; R1 is haloalkyl, R2 is heteroalkyl, and R3 is haloalkyl; R1 is haloalkyl, R2 is heteroalkyl, and R3 is heteroalkyl.

Class 13—R1 is heteroalkyl, R2 is hydrogen, and R3 is hydrogen; R1 is heteroalkyl, R2 is hydrogen, and R3 is alkyl; R1 is heteroalkyl, R2 is hydrogen, and R3 is haloalkyl; R1 is heteroalkyl, R2 is hydrogen, and R3 is heteroalkyl.

Class 14—R1 is heteroalkyl, R2 is alkyl, and R3 is hydrogen; R1 is heteroalkyl, R2 is alkyl, and R3 is alkyl; R1 is heteroalkyl, R2 is alkyl, and R3 is haloalkyl; R1 is heteroalkyl, R2 is alkyl, and R3 is heteroalkyl.

Class 15—R1 is heteroalkyl, R2 is haloalkyl, and R3 is hydrogen; R1 is heteroalkyl, R2 is haloalkyl, and R3 is alkyl; R1 is heteroalkyl, R2 is haloalkyl, and R3 is haloalkyl; R1 is heteroalkyl, R2 is haloalkyl, and R3 is heteroalkyl.

Class 16—R1 is heteroalkyl, R2 is heteroalkyl, and R3 is hydrogen; R1 is heteroalkyl, R2 is heteroalkyl, and R3 is alkyl; R1 is heteroalkyl, R2 is heteroalkyl, and R3 is haloalkyl; R1 is heteroalkyl, R2 is heteroalkyl, and R3 is heteroalkyl.

The compositions of the present invention comprise an asymmetric thiobarbituric acid-derived polymethine for optical measurement of membrane potentials. The fluorescent reagent comprises a moderately hydrophobic anion which is capable of redistributing from one face of a membrane to the other in response to changes in transmembrane potential. This anion is referred to as the mobile or moderately hydrophobic anion. The moderately hydrophobic ion is an anion which labels the extracellular face of the plasma membrane. Upon addition of the moderately hydrophobic fluorescent anion to the membrane, cell, or tissue preparation, the anion partitions into the plasma membrane, where it distributes between the extracellular and intracellular surfaces according to a Nernstian equilibrium. Changes in the membrane potential cause the fluorescent anion to migrate across the membrane so that it can continue to bind to whichever face (the intracellular or extracellular face) is now positively charged. The fluorescence intensity is a function of intracellular concentration of the hydrophobic ion and this concentration varies as the fluorescent anion redistributes back and forth across the membrane depending the membrane potential.

The measurement of fluorescence intensity provides a sensitive method for monitoring changes in the transmembrane potential. For example, if the membrane potential (intracellular relative to extracellular) changes from negative to positive, the fluorescent hydrophobic anion is pulled from the extracellular surface to the intracellular surface of the plasma membrane. This results in an increase in fluorescence intensity. Thus, fluorescence measurements at appropriate excitation and emission wavelengths provide a fluorescence readout which is sensitive to the changes in the transmembrane potential. Typically, the time constant for the redistribution of the fluorescent anion is rapid and in the millisecond to second time scale thus allowing the convenient measurement of both rapid cellular electrical phenomena such as action potentials or ligand-evoked channel opening and slower and more sustained changes evoked by altering the activity of ion pumps or exchangers.

On one aspect the detection method comprises: (a) introducing a first reagent comprising a moderately hydrophobic asymmetric polymethine anion capable of redistributing from a first face of the membrane to a second face of the membrane in response to changes in the potential of the membrane; (b) exposing the membrane to excitation light of appropriate wavelengths; (c) measuring fluorescence intensity of the polymethine indicator; and (d) relating the energy transfer to the change in plasma membrane potential.

Asymmetric thiobarbituric acid-derived polymethine oxonols serve as a voltage sensor and move within the membrane from one face of the membrane to another in response to changes in the transmembrane potential. The distribution of hydrophobic ions between the two membrane-aqueous interfaces (the extracellular interface and the intracellular interface) is determined by the membrane potential. Cations will tend to congregate at the negatively charged membrane interface and correspondingly, anions will move to the positively charged interface. The inherent sensitivity of the invention is based on the large interfacial concentration changes of the mobile ion at physiologically relevant changes in membrane potentials. The methods of this invention couple this change in interfacial concentration to an efficient fluorescence readout thus providing a sensitive method of detecting changes in transmembrane potential. The speed of the fluorescence change is dependent on the membrane translocation rate of the moderately hydrophobic ion.

One aspect of the present invention provides asymmetric thiobarbituric acid-derived anionic dyes which translocate across the membrane at much faster rates. The indicators of the present invention are also able to follow slower voltage changes over a time scale of seconds to minutes. It is generally preferred that the hydrophobic dye be an anionic species. Ester groups of biological membranes generate a sizable dipole potential within the hydrocarbon core of the membrane. This potential aids anion translocation through the hydrophobic layer but hinders cations. Therefore, where membrane translocation is concerned, anions have a tremendous inherent speed advantage over cations. For example, it is known that for the isostructural ions tetraphenylphosphonium cation and tetraphenylborate anion, the anion is much more permeable than the cation (Flewelling, R. F. and Hubbell, W. L. 1986. “The membrane dipole potential in a total membrane potential model”, Biophys. J. 49:541-552).

Preferably, the fluorescent ions which translocate across the plasma membrane are moderately hydrophobic in order to bind strongly to the plasma membrane and translocate rapidly across it in response to changes in transmembrane potential. Preferably, the ion will have a single charge which will be delocalized across a significant portion of the dye, preferably the entire dye. An oxonol's negative charge is distributed over the entire the chromophore. Compound 18 absorbs at 534 nm (ext. coefficient=190,000 M⁻¹ cm⁻¹), emits at 557 nm and has a quantum yield of >0.2 in octanol. Compound 3 translocates with a time constant <5 seconds in voltage clamped mammalian cells. Compound 5 absorbs at about 630 nm and emits at about 660 nm. The negative charge is further delocalized in such red-shifted oxonols. As expected, the translocation rates for the pentamethine oxonols are faster than for the trimethine oxonols. For example, delocalization of the charge reduces the Born charging energy (inversely proportional to anion radius) required to move a charged molecule from a hydrophilic to a hydrophobic environment and facilitates rapid translocation of ions (Benz, R. 1988. “Structural requirement for the rapid movement of charged molecules across membranes”, Biophys. J. 54:25-33). Increasing hydrophobicity minimizes release of the bound dye from the plasma membrane and buries the ion deeper into the membrane, which decreases the electrostatic activation energy for translocation. However, hydrophobicity cannot be increased without limit, because some aqueous solubility is required to permit cellular loading. This invention reveals that highly hydrophobic polymethine oxonols tend to give high assay background probably due to poor water solubility. If necessary, the oxonol dyes may be loaded with the aid of amphiphilic solubilizing reagents such as beta-cyclodextrin, Pluronics such as Pluronic F-127, or polyethylene glycols such as PEG400, which help solubilize the hydrophobic ions in aqueous solution. Polar groups on the ion should be kept to a minimum and shielded as much as possible to disfavor solvation in the head group region of the bilayer. In this invention, moderately hydrophobic thiobarbituric acid-derived polymethine oxonols are developed to have the optimized properties for optical measurement of membrane potentials.

An extremely useful property of these oxonols is that their fluorescence intensity is 5-30 times brighter when bound to membranes than in aqueous solution [Rink, T. J., Montecucco, C., Hesketh, T. R., and Tsien, R. Y. 1980. “Lymphocyte membrane potential assessed with fluorescent probes.” Biochim. Biophys. Acta 595, 15-30]. Furthermore, the negative charge is delocalized throughout the chromophore with the four equivalent oxygens containing the majority of the charge. The high electron affinity of the thiobarbiturate moieties discourages protonation, pKa<1, and resists photooxidative bleaching. The four N-alkyl groups and the thiocarbonyl give the molecule a necessary amount of hydrophobicity needed for tight membrane binding and rapid translocation.

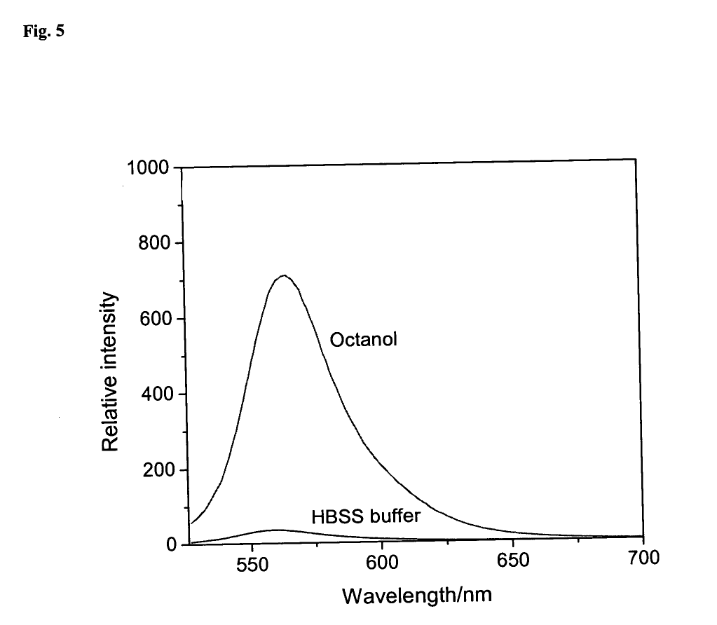

Preferably, the anions should be strongly fluorescent when adsorbed to the membrane, whereas they should have minimal fluorescence when free in aqueous solution. Preferably, the anionic fluorophores should be at least 2 times, and more preferably at least about 4 times, brighter when adsorbed to the membrane. In the case of the thiobarbituric oxonols described herein, their fluorescence is about 5-30 fold greater in the membrane than in water (see FIG. 5). In principle, if the dye bound extremely tightly to the membrane one would not need a high ratio of fluorescence when bound to the membrane to that when free in aqueous solution. However, it is desirable for the membrane potential-sensitive indicators to be at least about four times more strongly fluorescent in a membrane than in aqueous solution because in reality the volume of the membrane is tiny relative to the aqueous solution and some water solubility is necessary for loading of the dye into cells and tissue.

The anions also should not act as ionophores, especially protonophores, since such behavior may generate sustained leakage currents. Therefore, the protonation pKa of the anion is typically well below 7, preferably below 5, more preferably below 3. Red to infra-red wavelengths of excitation and emission are preferred to avoid tissue scattering and heme absorbances. Photodynamic damage should be kept as low as possible, probably best by minimizing triplet state formation and the resulting generation of singlet oxygen.

On another aspect of the present invention the detection method comprises: (a) introducing a first reagent comprising a moderately hydrophobic asymmetric polymethine anion capable of redistributing from a first face of the membrane to a second face of the membrane in response to changes in the potential of the membrane; (b) introducing a second reagent which labels the first face or the second face of the membrane, which second reagent comprises a fluorophore capable of undergoing energy transfer by either (i) donating excited state energy to the fluorescent anion, or (ii) accepting excited state energy from the fluorescent anion; (c) exposing the membrane to excitation light of appropriate wavelengths; (d) measuring energy transfer between the fluorescent anion and the second reagent; and (e) relating the energy transfer to the change in plasma membrane potential.

The preferred mode of energy transfer is fluorescence resonance energy transfer (FRET). The method finds particular utility in detecting changes in membrane potential of the plasma membrane in biological cells. The first and second reagents are spectroscopically complementary to each other, by which is meant that their spectral characteristics are such that excited state energy transfer can occur between them. Either reagent can function as the donor or the acceptor, in which case the other reagent is the corresponding complement, i.e., the acceptor or donor respectively. Both FRET and quenching are highly sensitive to the distance between the two species. For example, the non-radiative Forster-type quenching observed in FRET varies inversely with the sixth power of the distance between the donor and acceptor species. Therefore, when the membrane potential changes and the hydrophobic fluorescent anion moves either further away from or closer to the second reagent, FRET between the two reagents is either reduced or enhanced significantly. Other mechanisms such as electron-transfer, Dexter exchange interaction, paramagnetic quenching, and promoted intersystem crossing are even shorter-range and require the two reagents to collide or at least come within 1 nm of each other.

In one class of embodiments of the present invention, the moderately hydrophobic ion that fluorescences on one face of the membrane is quenched by a mechanism other than FRET. FRET has the advantages of working over long distances, which minimizes the necessary concentration of acceptors, and of giving ratiometric output at two emission wavelengths. However, if FRET is too efficient over very long distances greater than the thickness of the membrane, it can fail to discriminate between acceptors on the same vs. opposite sides of the membrane. The other mechanisms of quenching are much shorter-range and should never be effective across the thickness of the membrane.

FRET or fluorescence quenching is best detected by emission ratioing which can distinguish the two populations of the mobile fluorophore, i.e, those bound to the extracellular vs. those bound to the intracellular face of the membrane. In particular, FRET using a fluorescent acceptor provides an emission ratio change that is well suited to laser-scanning confocal microscopy and internally corrects for variations in donor loading, cell thickness and position (including motion artifacts), and excitation intensity. Emission ratios usually change by larger percentages than either emission wavelength signal alone, because the donor and acceptor emissions should change in opposite directions, which reinforce each other when ratioed. If emission ratioing is not desirable or possible, either wavelength can still be used alone, or the change in donor excited-state lifetime monitored.

High sensitivity is achieved when the voltage sensor, i.e., the polymethine oxonols, translocates at least a full unit charge nearly all the way through the membrane. Even without specific ion channels or transporters, such translocation can be quite rapid if the ion is negatively charged, delocalized, and moderately hydrophobic. However, voltage sensing should not require further diffusion of the ion through the unstirred aqueous layers that slows the response and generates a sustained leakage current.

To create an optical readout from the translocation of the moderately hydrophobic oxonol anion (i.e., the first reagent) from one side of the plasma membrane to the other side, FRET or fluorescence quenching between the translocating ion and a fluorophore or quencher (i.e., the second reagent) fixed to just one face of the plasma membrane is employed. Most conveniently, the extracellular face is employed.

Single cells were used in the examples so that the optical signals could be compared with voltage changes accurately known from traditional microelectrode techniques, such as patch clamping, which are applicable only to single cells. However, it should be apparent that the dyes can be used for many applications in which microelectrodes are not applicable. Comparison with microelectrodes is needed merely for accurate calibration and proof that the mechanism of fluorescence signal generation is as described herein. The reagent compositions and methods described herein can either resolve the different electrical potentials of many neighboring cells or neighboring parts of a single cell, or give an average reading for all the membrane locations, depending on whether the optical signal is spatially imaged or pooled.

The methods described herein are applicable to a wide variety of membranes. In particular, membrane potentials in membranes of biological cells can be detected and monitored. The method finds greatest utility with plasma membranes, especially the outermost plasma membrane of mammalian cells. Representative membranes include, but are not limited to, subcellular organelles, membranes of the endoplasmic reticulum, secretory granules, mitochondria, microsomes and secretory vesicles. Cell types which can be used include but are not limited to, neurons, cardiac cells, lymphocytes (T and B lymphocytes, nerve cells, muscle cells and the like.

The invention also provides methods for screening test samples such as potential therapeutic drugs which affect membrane potentials in biological cells. These methods involve measuring membrane potentials as described above in the presence and absence (control measurement) of the test sample. Control measurements are usually performed with a sample containing all components of the test sample except for the putative drug. Detection of a change in membrane potential in the presence of the test agent relative to the control indicates that the test agent is active. Membrane potentials can also be determined in the presence or absence of a pharmacologic agent of known activity (i.e., a standard agent) or putative activity (i.e., a test agent). A difference in membrane potentials as detected by the methods disclosed herein allows one to compare the activity of the test agent to that of the standard agent. It will be recognized that many combinations and permutations of drug screening protocols are known to one of skill in the art and they may be readily adapted to use with the method of membrane potential measurement disclosed herein to identify compounds which affect membrane potentials.

Use of the membrane potential determination technique disclosed herein in combination with all such methods are contemplated by this invention. In a particular application, the invention offers a method of identifying a compound which modulates activity of an ion channel, pump, or exchanger in a membrane, comprising: (a) loading the cells with the asymmetric thiobarbituric acid-derived polymethine oxonols; (b) determining the membrane potential as described above; (c) exposing the cells to the test sample; (d) redetermining the membrane potential and comparing with the result in (b) to determine the effect of the test sample; (e) optionally, exposing the membrane to a stimulus which modulates an ion channel, pump or exchanger, and redetermining the membrane potential and comparing with the result in (d) to determine the effect of the test sample on the response to the stimulus.

In another application, the invention offers a method of screening test samples to identify a compound which modulates the activity of an ion channel, pump or exchanger in a membrane, comprising: (a) loading a first set and a second set of cells with the voltage-sensitive oxonols which measure membrane potential; (b) optionally, exposing both the first and second set of cells to a stimulus which modulates the ion channel, pump or exchanger; (c) exposing the first set of cells to the test sample; (d) measuring the membrane potential in the first and second sets of cells; and (e) relating the difference in membrane potentials between the first and second sets of cells to the ability of a compound in the test sample to modulate the activity of an ion channel, pump or exchanger in a membrane.

Ion channels of interest include, but are not limited to, sodium, calcium, potassium, nonspecific cation, and chloride ion channels, each of which may be constitutively open, voltage-gated, ligand-gated, or controlled by intracellular signaling pathways.

Biological cells which can be screened include, but are not limited to primary cultures of mammalian cells, cells dissociated from mammalian tissue, either immediately or after primary culture. Cell types include, but are not limited to white blood cells (e.g. leukocytes), hepatocytes, pancreatic beta-cells, neurons, smooth muscle cells, intestinal epithelial cells, cardiac myocytes, glial cells, and the like. The invention also includes the use of recombinant cells into which ion transporters, ion channels, pumps and exchangers have been inserted and expressed by genetic engineering. Many cDNA sequences for such transporters have been cloned (see U.S. Pat. No. 5,380,836 for a cloned sodium channel) and methods for their expression in cell lines of interest are within the knowledge of one of skill in the art (see, U.S. Pat. No. 5,436,128). Representative cultured cell lines derived from humans and other mammals include LM (TK.sup.-) cells, HEK293 (human embryonic kidney cells), 3T3 fibroblasts, COS cells, CHO cells, RAT1 and HLHepG2 cells.

The screening methods described herein can be made on cells growing in or deposited on solid surfaces. A common technique is to use a microtiter plate well wherein the fluorescence measurements are made by commercially available fluorescent plate readers. The invention includes high throughput screening in both automated and semiautomated systems. One such method is to use cells in Costar 96 well microtiter plates (flat with a clear bottom) and measure fluorescent signal with CytoFluor multiwell plate reader (Perseptive Biosystems, Inc., MA) using a single wavelength to read fluorescence intensity or two emission wavelengths to record fluorescent emission ratios.

### DETAILED DESCRIPTION OF THE INVENTION

The invention may be better understood with reference to the accompanying examples, which are intended for purposes of illustration only and should not be construed as in any sense limiting the scope of the invention as defined in the claims appended hereto.

### EXAMPLE 1

### Synthesis of Compound 11

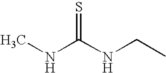

For all the syntheses, all starting materials and reagents were of the highest purity available (Aldrich Chemical Company, Milwaukee, Wis.) and used without further purification, except where noted. Solvents were HPLC grade (Fisher Scientific, Pittsburgh, Pa.) and were dried over activated molecular sieves. NMR spectra were acquired on a Varian Gemini 200 MHz spectrometer. Absorption and fluorescence spectra were taken respectively on a Cary 50 BIO and Varain eClipse (Varian, Inc., Palo Alto, Calif.).

To 2 M ethylamine in THF (200 ml, 0.4 mol) methyl isothiocyanate (29.25 g, 0.4 mol) in THF (60 mL) is added proportionwise under stirring in an ice/water bath. The reaction mixture is stirred at room temperature overnight, and TLC (hexane/ethyl acetate=1/1) is used to confirm the completion of the reaction. Solvent is removed in-vacuo and the residue is further dried under high vacuum to give a syrup. The residue is then mixed with hexane 300 (mL) and is stirred for 1 hour to give an off-white precipitate. The solid is collected by filtration, and washed with hexane (2×50 ml) to give an off-white solid (46 g, 97%). Rf=0.23 (hexane/ethyl acetate=1/1).

### EXAMPLE 2

### Synthesis of Compound 12

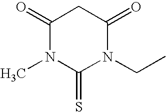

To anhydrous methanol (160 mL) is added sodium (9.2 g, 0.4 mol) in small piece with a setup of cooling condenser. During the addition the reaction mixture is spontaneously heated to reflux. After the sodium pellets are completely consumed, to the reaction mixture is added diethyl malonate (60.8 mL, 0.4 mol) in one portion, followed by adding N-ethyl-N′-methyl thiourea (23.64 g, 0.2 mol). The reaction mixture is refluxed overnight (22 h) and TLC (chloroform/methanol=7/3) is used to confirm the completion of the reaction. The solvent is removed in-vacuo, and the residue is dissolved in water (150 mL). The aqueous solution is acidified with 32% HCl to pH=2 under cooling. The formed light yellow solid is collected by filtration and air-dried. The dry solid is washed with hexane/ethyl acetate (1/1) until a white solid is obtained (22.5 g, 60%). Rf=0.36 (chloroform/methanol=7/3).

### EXAMPLE 3

### Synthesis of Compound 13

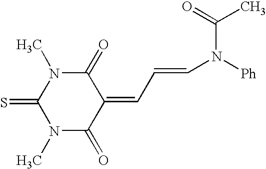

N,N′-Dimethyl thiobarbituric acid (1.72 g, 10 mmol), malonaldehyde dianil hydrochloride (2.59, 10 mmol) and sodium acetate (0.82 g, 10 mmol) in acetic andydride (15 mL) is refluxed for 20 min in an oil bath. The reaction mixture is cooled in an ice/water bath. To the formed precipitate is added 1:1 cold water/methanol (40 mL), and stirred. The precipitate is collected by filtration and then washed with methanol (3×10 mL) to give a dark brown solid that is dried under high vacuum to give the desired product (3.1 g).

### EXAMPLE 4

### Synthesis of Compound 14

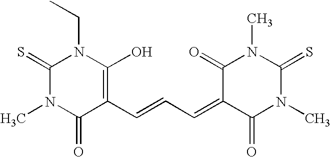

Compound 13 (3.1 g, 9 mmol) is suspended in acetonitrile (20 mL), and to the suspension N-ethyl-N′-methyl thiobarbituric acid (1.68 g, 9 mmol) is added. To the reaction mixture is then added triethylamine (6.3 ml, 45 mmol), and stirred at room temperature for 4 h. TLC is used to confirm the completion of reaction. The formed purple precipitate is collected by filtration and washed with acetonitrile (3×5 mL). The crude product is further purified by column chromatorgraphy using a gradient of chloroform/methanol to give the desired product (2.5 g, 70%). Rf=0.62 (chloroform/methanol/triethylamine=80/15/5).

### EXAMPLE 5

### Synthesis of Compound 15

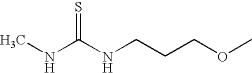

3-Methoxypropylamine is reacted with methylisothiocynate according to the procedure of Compound 11 to give the desired product.

### EXAMPLE 6

### Synthesis of Compound 16

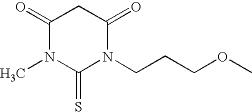

Compound 15 is reacted with dietyl malonate according to the procedure of Compound 12 to give the desired product.

### EXAMPLE 7

### Synthesis of Compound 17

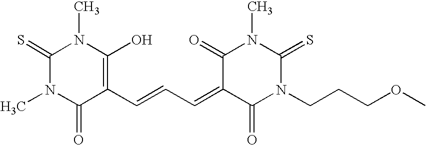

Compound 17 is prepared from Compounds 13 and 16 analogous to the procedure of Compound 14.

### EXAMPLE 8

### Synthesis of Compound 18

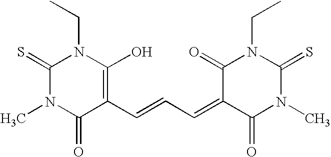

Compound 12 (1 g, 5.37 mmol) and malonaldehyde dianil hydrochloride (695 mg, 2.69 mmol) are dissolved in acetonitrile (8 mL). To this solution, triethylamine (1.46 g, 14.4 mmol) is added. The solution immediately turned red. After 3 h, the reaction mixture is concentrated, and formed precipitate is collected by filtration. The desired product (267 mg) is collected after washing the crystals with acetonitrile and then drying under high vacuum.

### EXAMPLE 9

### Synthesis of Compound 19

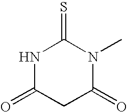

N-methylthiourea is reacted with diethyl malonate according to the procedure of Compound 12 to give the desired product.

### EXAMPLE 10

### Synthesis of Compound 20

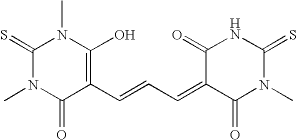

Analogous to the procedure of Compound 14, Compound 13 is suspended in acetonitrile, and to the suspension Compound 19 is added. To the reaction mixture is then added triethylamine, and stirred at room temperature for 4 h. TLC is used to confirm the completion of the reaction. The formed purple precipitate is collected by filtration and washed with acetonitrile. The crude product is further purified by column chromatography using a gradient of chloroform/methanol (as an eluant) to give the desired product.

### EXAMPLE 11

### Synthesis of Compound 21

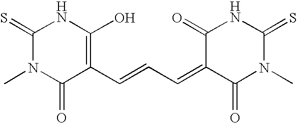

Analogous to the procedure of compound 18, Compound 19 and malonaldehyde dianil hydrochloride are dissolved in acetonitrile. To this solution, triethylamine is added. The solution immediately turned red. After 3 h, the reaction mixture is concentrated, and formed precipitate is collected by filtration. The desired product is collected after washing the crystals with acetonitrile and then drying under high vacuum.

Other oxonols are made as illustrated in FIG. 3. Specifically the required barbiturates are prepared from the appropriate thiourea either prepared from required primary amine and carbon disulfide [Bortnick, N., Luskin, L. S., Hurwitz, M. D., and Rytina, A. W. 1956. t-Carbinamines, RR′R″CNH₂. III. The preparation of isocyanates, isothiocyanates and related compounds. J. Am. Chem. Soc. 78:4358-4361] or from the reactions of thiophosgen with the amines. The barbiturates are converted to the desired polymethine oxonols through the imine intermediates of Structure II.

### EXAMPLE 12

### Water Solubility and Hydrophobicity Comparison of DiBAC and DiSBAC Dyes

The thiobarbituric acid-derived polymethine oxonols are dissolved in DMSO (3 mM). The DMSO stock solutions are respectively partitioned in 1:1 octanol/HBSS buffer mixture. The concentrations of the oxonol dyes in octanol and aqueous layers are determined by absorption spectra. This invention concludes that the moderately hydrophobic compounds give the highest quality of assay data for measuring membrane potentials in cells.

### EXAMPLE 13

Measuring membrane potentials using Compound 14, 17, 18, 20 or 21 P2X2 belongs to a class of purinergic ion channels that pass calcium and sodium in response to purine, including adenosine 5′-triphosphate (ATP). The cells are 1321 N1 astrocytoma cells transfected to overexpress the purinergic P2X2 ligand-gated ion channel. Adapted from the published procedures [Baxter D F, Kirk M, Garcia A F, Raimondi A, Holmqvist M H, Flint K K, Bojanic D, Distefano P S, Curtis R, Xie Y. “A novel membrane potential-sensitive fluorescent dye improves cell-based assays for ion channels.” J Biomol Screen 7, 79 (2002); Falconer M, Smith F, Surah-Narwal S, Congrave G, Liu Z, Hayater P, Ciaramella G, Keighley W, Haddock P, Waldron G, Sewing A. “High-throughput screening for ion channel modulators” J Biomol Screen 7, 460 (2002)], P2X2 cells are propagated and maintained in DME (high glucose), 10% FCS, Pen/Strep and 2 mM L-glutamine. The P2X2 cells are split at a 1 to 2 ratio upon confluence. 40,000 P2X2 cells are plated in 100 μL per well for 96 well plates or 10,000 P2X2 cells are plated in 25 μL per well for 384 well plates for overnight. Compound 14, 17, 18, 20 or 21 is dissolved in DMSO. The DMSO stock solution is diluted with 20 mM HEPES (pH 7.40), incubated with the cells in HBSS buffer. The assay plates are incubated at 37° C. for 30-45 minutes. The fluorescence changes are recorded with a fluorescence microplate reader (preferably with an integrated liquid handling system), a microscope or a flow cytometer. An initial depolarization event is depicted as an increase in fluorescence followed by repolarization or decay in signal near baseline. EC50 should be in the range of 10-100 nM.

### EXAMPLE 14

### Measurement of Membrane Potential with Compound 14, 17, 18, 20 or 21 as FRET Acceptors and Fluorescent Lectins as FRET Donors

Compound 14, 17, 18, 20 or 21 is used to measure transmembrane potential in a FRET mode analogous to the procedure as described by Adkins C E, G V, Kerby J, Bonnert T P, Haldon C, McKeman R M, Gonzalez J E, Oades K, Whiting P J and Simpson P B. [“alpha4beta3delta GABA(A) receptors characterized by fluorescence resonance energy transfer-derived measurements of membrane potential.” J Biol Chem 276, 38934-9 (2001)].

### EXAMPLE 15

### Detection of cAMP-Dependent Activation of Cyclic Nucleotide-Gated Channels (CNG Channels) Using Compound 14, 17, 18, 20 or 21

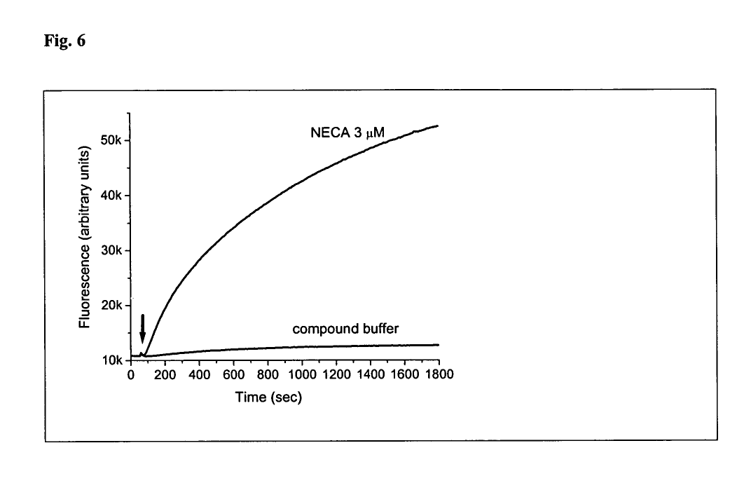

The HEK293 cell line that stably incorporates a CNG channel mutant gene is prepared, and then loaded with Compound 14, 17, 18, 20 or 21 by incubating the cells for 2 hours at room temperature with the dye at 5 μM, in combination with a membrane-impermeable non-fluorescent dye to quench extracellular fluoescence (Sahlin S, Hed J, Rundquist I. 1983. “Differentiation between attached and ingested immune complexes by a fluorescence quenching cytofluorometric assay.” J. Immunol. Methods 60:115-124). The CNG channels are activated by cyclic nucleotides (cGMP and cAMP), whereby the conducting cation currents are carried by mixed ions Na⁺, K⁺ and Ca²⁺, and the changes in intracellular cyclic nucleotide concentration are coupled to membrane potential changes (Yao Y and Cao L. “Novel cell-based assays for G-protein-coupled receptor-mediated activities.” U.S. Patent Application, 20030100059). [cAMP]ᵢ increase is detected by a membrane depolarization signal through CNG channels. The kinetic responses are recorded on a fluorescence plate reader, e.g., FLIPR (Molecular Devices Corp., Sunnyvale, Calif.). Activation of one endogenous G protein-coupled adenosine A2B receptor is detected by measuring membrane depolarization mediated through activation of CNG channels (see FIG. 6). Addition of NECA agonist at 3 μM (marked with an arrow in FIG. 6) affords the increase in fluorescence intensity of Compounds 14, 17, 18, 20 or 21 by about 2-10 fold.

### EXAMPLE 16

### Comparison of Compound 14, 18, 20 or 21 with Symmetric Thiobarbituric Acid-Derived Polymethine Oxonols for Measuring Membrane Potentials

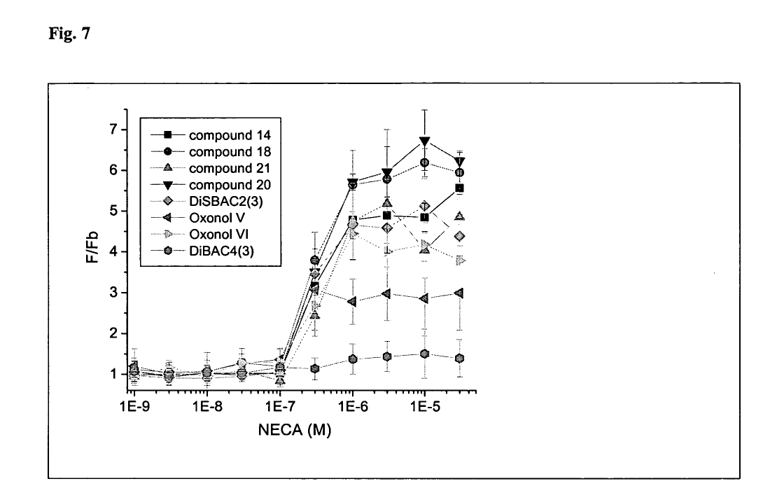

DiSBAC₂(3), DiBAC₄(3), oxonol V, and oxonol VI are the most common existing fluorescent dyes used for determining CNG channel activity, and their performances for measuring membrane potentials are compared with asymmetric moderately hydrophobic thiobarbituric acid-derived polymethine oxonols of this invention using the CNG channel activity as an assay model. Specifically, the CNG channel is activated by NECA in a dose-dependent manner. Endpoint assays are performed in 384-well plates. A stable HEK293 cell line expressing one CNG channel mutant is used in the assays. Cells are loaded with different voltage-sensitive dyes for about 2 hours at room temperature. Fluorescence change expressed in signal over the background (F/Fb) is measured 30 minutes after addition of NECA at various concentrations. Dose-responses of NECA are plotted as shown in FIG. 7. The moderately hydrophobic asymmetric thiobarbituric acid-derived polymethine oxonols of this invention, e.g., Compounds 18 and 20 demonstrate significant improvement in fluorescence signal strength and in quality of assay data over the existing symmetric thiobarbituric acid-derived polymethine oxonols.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A method for measuring transmembrane potential changes in a biological system using a composition comprising a polymethine oxonol compound of Structure I as a fluorescent potentiometric indicator. 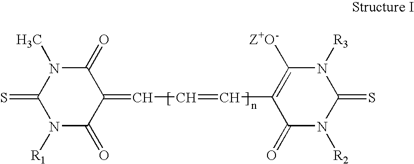
wherein R1, R2, and R3 are (a) independently selected from the group consisting of hydrogen, alkyl, haloalkyl and heteroalkyl, and (b) R1, R2 and R3 are not simultaneously methyl; n is an integer from 1 to 3; Z is Na, K, ammonium or other biologically acceptable salt.

2. The composition of claim 1, wherein said R1 is hydrogen, R2 and R3 are respectively methyl, ethyl or alkyl group, haloalkyl or heteroalkyl of lesss than 12 carbons or independently selected from the group consisting of hydrogen, methyl, ethyl or alkyl group, haloalkyl and heteroalkyl.

3. The composition of claim 2, wherein said R1 is hydrogen, R2 and R3 are methyl or ethyl.

4. The composition of claim 1, wherein said R1 and R2 are hydrogen, R3 are methyl, ethyl, or alkyl group, haloalkyl or heteroalkyl of lesss than 12 carbons.

5. The composition of claim 4, wherein said R1 and R2 are hydrogen, R3 is methyl or ethyl.

6. The composition of claim 1, wherein said R1 is methyl, R2 and R3 are ethyl, or alkyl group, haloalkyl or heteroalkyl of lesss than 12 carbons or independently selected from the group consisting of hydrogen, R2, R3 and R3 are respectively methyl, ethyl or alkyl group, haloalkyl and heteroalkyl.

7. The composition of claim 6, wherein said R1 and R2 are ethyl, R3 is methyl.

8. The method of claim 1, wherein the assay system comprises: (a) a fluorescent anion of Structure I redistributes from one side of a membrane of said biological system to a second side of said membrane in response to electrical potential across said membrane; (b) stimulating membrane potential changes physically or with a biologically active substance; (c) measuring the fluorescence changes; and (d) correlating the fluorescence signal to change in membrane potentials.

9. The method of claim 1, wherein the potentiometric probe is used to measure transmembrane potential changes in combination with a second fluorescent reagent.

10. The method of claim 9, wherein a compound of Structure I is used in combination with a second fluorescent indicator so that there is fluorescence energy transfer (FRET) between the two fluorescent reagents.

11. The method of claim 10, wherein the measurement is performed comprising: (a) a first reagent comprising a compound of Structure I from one side of a membrane to a second side of said membrane in response to potential across said membrane, (b) a second reagent, comprising a fluorescent compound that is a FRET partner to said first reagent, and (c) wherein said first FRET partner and said second FRET partner exhibits a change in FRET in response to a change in transmembrane potential.

12. The method of claim 11, wherein the fluorescence intensity ratio of the first reagent to the second reagent is recorded.

13. The method of claim 12, wherein the fluorescence intensity ratio of the first reagent to the second reagent is correlated to changes in membrane potential of a biological system.

14. The method of claim 9, wherein the second fluorescent reagent is a derivative of coumarins, fluoresceins, rhodamines, carbocyanines, BODIPYs, polycyclic aromatic compounds, or lanthanide complex.

15. The composition of claim 9, wherein said second reagent comprises a fluorescently labeled peptide, protein, nucleotide, nucleic acid, carbohydrate, lectin, lipid, phospholipid, cytochrome or antibody.

16. The method of claim 9, wherein the second fluorescent reagent is a fluorescent protein that is expressed within said living cell.

17. The method of claim 1, wherein a fluorescent compound of Structure I is used in combination with a second non-fluorescent quenching compound so that the fluorescence intensity of said compound of Structure I is dependent on the non-fluorescent quenching compound.

18. The method of claim 1, wherein the membrane is a plasma membrane of a biological cell.

19. The method of claim 1, wherein the membrane is a mitochondron membrane of a biological cell.

20. The method of claim 1, wherein the measurement is made in a fluorescence microplate reader, a fluorescence flow cytometer, or a fluorescence microscope.

21. A test kit according to claim 1 for measuring membrane potential changes comprising a compound of structure I as a reagent.

22. The kit of claim 21, further comprising a solubilizing agent.

23. A test kit according to claim 10 for measuring membrane potential changes comprising a compound of structure I and a second fluorescent reagent.

24. A test kit according to claim 17 for measuring membrane potential changes comprising a compound of structure 1 and a second non-fluorescent colored reagent.

25. A process of preparing membrane potential-sensitive fluorescent indicators of Structure by using intermediates of Structure II. 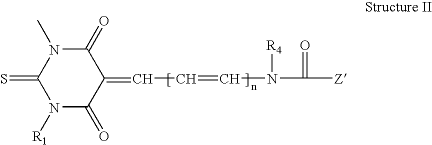
wherein R1 and R4 are independently selected from the group consisting of hydrogen, alkyl, haloalkyl and heteroalkyl; n is an integer from 1 to 3; Z′ is a hydrocarbyl group.

26. According to claim 25, wherein said Z′ is methyl or trifluoromethyl.

27. According to claim 25, wherein said R4 is phenyl.
